(19) United States
(12) Patent Application Publication
(10) Pub. No.: US 2025/0333389 A1
(43) Pub. Date: Oct. 30, 2025

(54) PHARMACEUTICAL FOR THE INHIBITION OF CELLULAR PROTON PUMPS

(71) Applicant: AquaRemedi Pharmaceuticals INC, Newport Beach, CA (US)

(72) Inventor: Mohamad Kourghi, Newport Beach, CA (US)

(21) Appl. No.: 18/647,940

(22) Filed: Apr. 26, 2024

(51) Int. Cl. C07D 277/04; A61K 31/426; C07D 277/06

(52) U.S. Cl. CPC C07D 277/04; A61K 31/426; C07D 277/06

(57) ABSTRACT

The present invention provides technologies relating to modulation of voltage-gated ion channels. Particularly, the present invention provides pharmaceutical compounds for inhibiting ion transport by a voltage-gated ion channel in a subject in need thereof. In certain embodiments of the invention, the ion is a proton and the voltage-gated ion channel is a voltage gated proton channel such as Hv1. The invention further provides methods of administering a therapeutically effective amount of the pharmaceutical compounds to a subject to block the proton transport by a voltage-gated proton channel such as Hv1.

### FIELD OF THE INVENTION

The present invention relates to the fields of molecular biology and drug discovery, and particularly relates to pharmaceutical compounds for the inhibition of cellular proton pumps such as Hv1 voltage-gated ion channels. Certain embodiments of the present invention relate to small molecule modifiers of Hv1 ion channel transport activity, and uses thereof.

### BACKGROUND

Voltage-gated ion channels facilitate the transfer of ions across cell membranes and function as key components of essential cellular processes. One particular type of voltage-gated ion channel is the voltage-gated proton channel (Hv1). Hv1 is a transmembrane protein that regulates the transfer of protons across cell membranes. When the Hv1 channel is open, protons permeate the channel and cross the cellular membrane.

Hv1 channels are expressed in different tissues and are associated with a wide variety of physiological and pathological processes. For example, Hv1 channels may play a role in immune defense, sperm activity, and cancer progression. For these and other reasons, Hv1 is a compelling target for discovery of corresponding inhibitor drugs. (See Seredenina, T., et al. “Voltage-gated proton channels as novel drug targets: from NADPH oxidase regulation to sperm biology.” Antioxid Redox Signal. 10; 23 (5): 490-513 (2015)). Hv1 voltage-gated proton channels have been shown to play important roles in proton extrusion, pH homeostasis, and production of reactive oxygen species in a variety of cell types. Hv1 has been found to be highly expressed in breast cancer cells; and its knockdown by RNA interference has been shown to strongly reduce cell proliferation and invasiveness. Hv1 activity has also been shown to be involved in NOX-mediated neuronal death during cerebral ischemia; and mice lacking Hv1 activity have been shown to be protected from brain damage after stroke. (See U.S. Patent Publication No. 20150203812).

Due to the significant physiological consequences of blocking of voltage-gated proton channels, several studies have been conducted to discover effective activators or blockers of Hv1 channel. For example, Zhao et al. developed C6, a specific peptide inhibitor of hHv1, to evaluate the roles of the channel in sperm capacitation and in the inflammatory immune response of neutro-phils. (R. Zhao et al., Role of human Hv1 channels in sperm capacitation and white blood cell respiratory burst established by a designed peptide inhibitor, Proc. Natl. Acad. Sci. U.S.A. 115, E11847-E11856, 2018). Zhao further indicated that the C6 can inhibit voltage-gated proton channels (Hv1) and demonstrated that it can suppress the release of reactive oxygen species (ROS) and proteases from neutrophils in vitro. Zhao also showed that intravenous C6 counteracts bacterial lipopolysaccharide (LPS)-induced ALI in mice, and suppresses the accumulation of neutrophils, ROS, and proinflammatory cytokines in bronchoalveolar lavage fluid. (Zhao et al., Protection from acute lung injury by a peptide designed to inhibit the voltage-gated proton channel, iScience, 2022, doi: 10.1016/j.isci.2022.105901, PMID: 36660473, PMCID: PMC9843441). Using C6, it is also shown that the human voltage-gated proton channel (hHv1) is the main pathway for H+ efflux that allows capacitation in sperm and permits sustained reactive oxygen species (ROS) production in white blood cells (WBCs). (R. Zhao et al., Role of human Hv1 channels in sperm capacitation and white blood cell respiratory burst established by a designed peptide inhibitor, Proc. Natl. Acad. Sci. U.S.A. 115, E11847-E11856, 2018).

The proton-pump inhibitor drugs (PPIs) have been also widely used in acid-related disorders. For example, PPIs have been used for reducing stomach acid levels. PPIs have been further used for preventing and treating ulcers in the stomach and duodenum.

Based on the foregoing, it is an object of the present invention to provide effective synthetic compounds/blockers for the inhibition of cellular proton pumps, and more particularly, to provide synthetic pharmaceutical compounds for the inhibition of Hv1 (hHv1) channel for the treatment of various kinds of diseases such as Alzheimer's disease. These and other objects are more fully described in the following specification and drawings.

### SUMMARY

The present disclosure provides technologies relating to modulation of voltage-gated ion channels. Among other things, the present invention provides chemical compounds, blockers, modulating agents, or related compositions and methods for inhibiting ion transport by a voltage-gated ion channel in a subject in need thereof. In certain embodiments of the invention, the ion is a proton and the voltage-gated ion channel is a voltage gated proton channel such as Hv1. In some embodiments, the subject comprises a plurality of cells that express the voltage-gated proton channel. The methods involve administering to the subject a therapeutically effective amount of a the compound that is capable of inhibiting proton transport by the voltage-gated proton channel.

Certain embodiments of the present invention provide compounds that synthesized from marine fungi that are capable of modifying proton transport in a voltage-gated ion channel such as the Hv1 voltage-gated proton channel. The disclosed compounds of the present invention are characterized by having a property of inhibiting ion transport by the voltage-gated ion channel, such as inhibiting proton transport by Hv1. As the original marine fungi is a very weak blocker of Hv1 channels, the inventor used the marine fungi as a backbone to design more potent Hv1 channel blockers. In particular, an embodiment of the invention provides a substituent compound (S1) S1 that synthesized from the original marine fungi compound. Another embodiment of the invention provides another substituent compound (S2) that is synthesized from S1 by removing the extra side chain of the substituent S1 that is not involved in binding with the Hv1 channel.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a molecule structure of a compound (“Original Compound”) obtained from marine fungi.

FIG. 2 illustrates representative proton current traces for Hv1 channels before (Left in black), and in the presence of 10 μM of the Original Compound (Right in red).

FIG. 3 illustrates a current-voltage relationships (I-V) for Hv1 in the absence (black) or presence of 10 μM of the Original Compound (red).

FIG. 4 illustrates a conductance-voltage relationships (G-V) for Hv1 in the absence (black) or presence of 10 μM of the Original Compound (red).

FIG. 5 illustrates a first substituent compound (S1) that is synthesized from the Original Compound.

FIG. 6 illustrates representative proton current traces for Hv1 channels before (Left in black), and in the presence of 100 nM of S1 (Right in red).

FIG. 7 illustrates a current-voltage relationships (I-V) for Hv1 in the absence (black) or presence of 100 nM of S1 (red).

FIG. 8 illustrates a conductance-voltage relationships (G-V) for Hv1 in the absence (black) or presence of 100 nM of S1 (red).

FIG. 9 illustrates a time course for block and unblock of Hv1 when S1 is applied and when S1 is washed away.

FIG. 10 illustrates a Cryogenic-Electron Microscopy data indicating how the S1 compound interacts with the Hv1 channel.

FIG. 11 illustrate a side chain on S1 that is not directly involved in interacting with Hv1 (highlighted in red circle).

FIG. 12 illustrates a second substituent compound (S2) that is synthesized from the Original Compound.

FIG. 13 illustrates representative proton current traces for Hv1 channels before (Left in black), and in the presence of 100 nM of S2 (Right in red).

FIG. 14 illustrates a current-voltage relationships (I-V) for Hv1 in the absence (black) or presence of 100 nM of S2 (red).

FIG. 15 illustrates a conductance-voltage relationships (G-V) for Hv1 in the absence (black) or presence of 100 nM of S2 (red).

### DESCRIPTION

The present invention is described more fully hereinafter, but not all embodiments are shown. While the invention has been described with reference to exemplary embodiments, it will be understood by those skilled in the art that various changes may be made, and equivalents may be substituted for elements thereof without departing from the scope of the disclosure. In addition, many modifications may be made to adapt a particular structure or material to the teachings of the disclosure without departing from the essential scope thereof.

The drawings accompanying the application are for illustrative purposes only. They are not intended to limit the embodiments of the present application. Additionally, the drawings are not drawn to scale. Common elements between different figures may retain the same numerical designation.

As described in detail below, various embodiments of the present invention provide efficient Hv1 channel blockers and related methods of administration for the treatment of various kinds of diseases. In some embodiments, the strength of such proton transport inhibition is in a range selected from about 10% or more, 20% or more, 30% or more, 40% or more, 50% or more, 60% or more, 70% or more, 80% or more, and 90% or more.

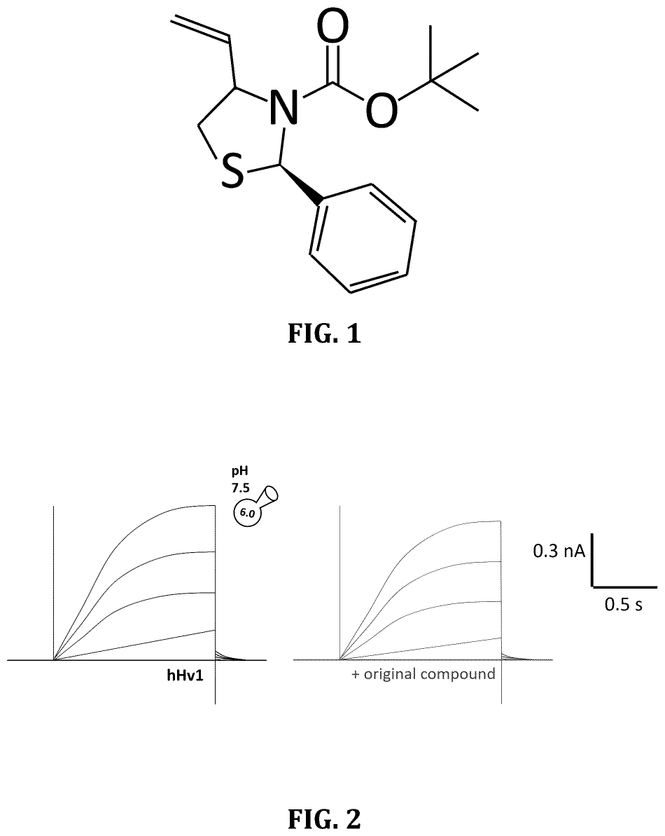

FIG. 1 illustrates a molecule structure of a compound (“Original Compound”) obtained from marine fungi, having a chemical formula of (2R,4R)-tert-butyl 2Phenyl-4-vinylthiazolidine-3-carboxylate.

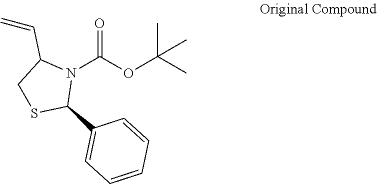 [SMILES: C=CC1CS[C@H](c2ccccc2)N1C(=O)OC(C)(C)C]

As illustrated in FIG. 2, the Original Compound is known as a weak pore blocker of the human Hv1 channel. FIG. 2 illustrates representative proton current traces for Hv1 (hHv1) channels before (Left in black), and in the presence of 10 μM concentration of the Original Compound (Right in red). The proton current traces was monitored by evoking currents from a holding voltage of −60 mV with stepping to 0 mV for 1.5 s with a 10 s interpulse interval. As shown in FIG. 2, the Original Compound at 10 μM concentration blocks only 20% of Hv1 current at −20 mV.

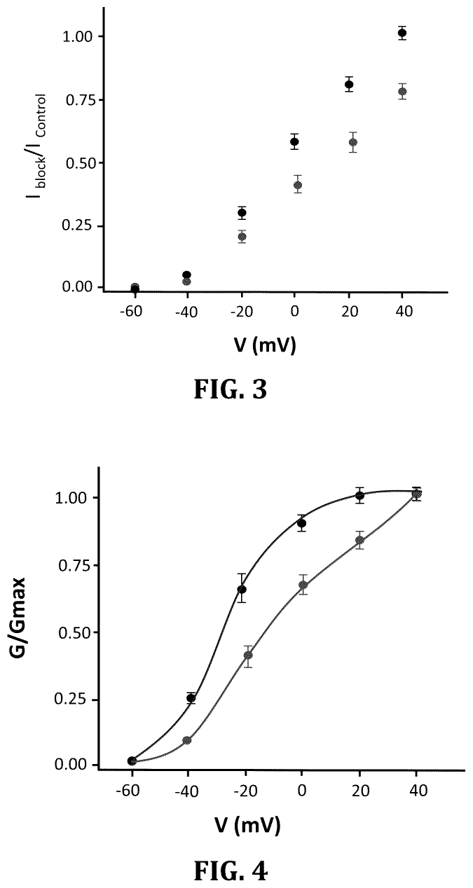

FIG. 3 illustrates a current-voltage relationships (I-V) for Hv1 in the absence (black) or presence of 10 μM of the Original Compound (red). The current-voltage relationships were evoked from a holding potential of −60 mV to test pulses from −60 mV to +40 mV for 1.5 s in 20 mV intervals every 10 s. The fractional unblocked current was assessed at the end of the test pulse. From this figure, we observe that at −20 mv and in the presence of 10 μM concentration of the Original Compound, we only obtain 20% proton current reduction.

The inhibitor constant of the Original Compound is further calculated to examine the effectiveness of the compound in inhibiting proton currents in Hv1 channels. The inhibitor constant, Ki, is the concentration required to produce half maximum inhibition which is an indication of how potent an inhibitor is. In other words, the smaller the Ki the greater the binding affinity and the smaller the amount of compound is needed to inhibit the activity of the channel. The inhibition constant Ki of the Original Compound for Hv1 channels was calculated as Ki=Kon/Koff, wherein Kon was 3.2*10\hat{ }5, Koff was 1.05*10\hat{ }5, and accordingly, Ki was estimated to be 3 μM. Inhibition constant Ki can also be calculated based on the Hill equation, in which Ki=[Tx]/(1−Fun)/Fun. TX is the compound concentration, and Fun is the fractional unblock current.

The conductance-voltage relationships of Hv1 channels in the absence and presence of the Original Compound further confirms that the Original Compound is a weak blocker of the Hv1 proton current. FIG. 4 illustrates a conductance-voltage relationships (G-V) for Hv1 channel in the absence (black) or presence of 10 μM of the Original Compound (red). The G-V curve shows about only 15 mV V1/2 shift to the left, indicating that the Original Compound is a weak pore blocker for the Hv1 channel. The conductance-voltage relationships were fit to the Boltzmann equation, G=Gmax/[1+exp(−zF(V−V1/2)/RT)], where V is the test potential, V1/2 is the voltage of half-maximal activation, z is the effective valence, T is the temperature, R is the gas constant, and F is the Faraday constant.

### First Substituent Compound (S1)

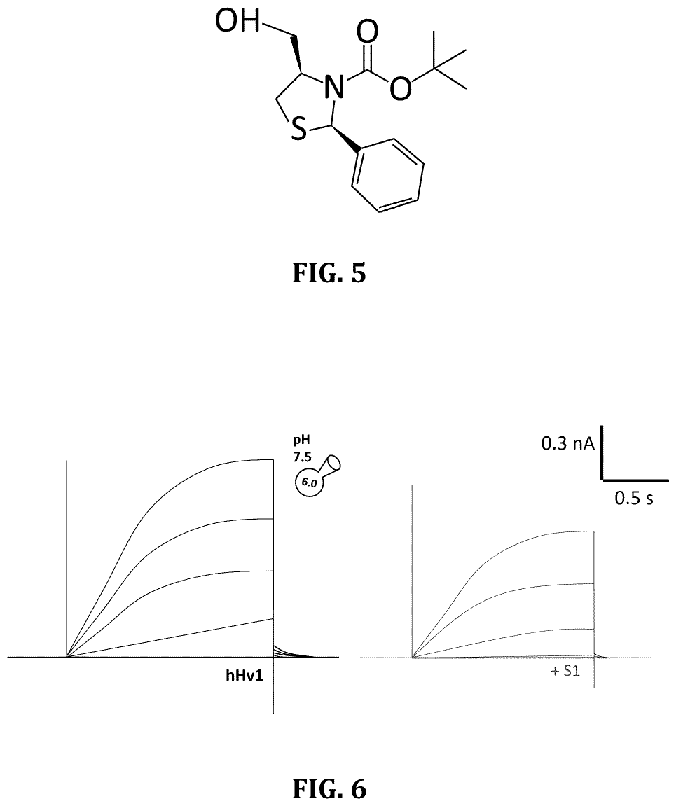

Based on observed properties of the Original Compound (as shown in FIG. 1), by modifying and altering the side chains of the Original Compound, Inventor developed strong pore blockers/inhibitors of Hv1 channels including a first substituent compound (S1) as shown in FIG. 5 and a second substituent compound (S2) as shown in FIG. 12. The first substituent compound (S1) can be synthesized from the Original Compound. S1 has a molecule structure as illustrated in FIG. 5 (and below), and has a chemical formula of (2R,4R)-tert-butyl 4-(hydroxymethyl)-2-Phenylthiazolidine-3-carboxylate.

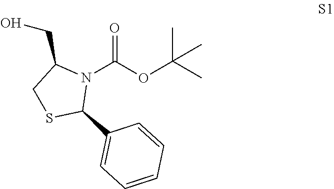 [SMILES: CC(C)(C)OC(=O)N1[C@H](CO)CS[C@@H]1c1ccccc1]

The first substituent S1 (shown in FIG. 5) can be made by changing the orientation of the active side chain of the Original Compound (shown in FIG. 1).

FIG. 6 illustrates representative proton current traces for Hv1 (hHv1) channels before (Left in black), and in the presence of 100 nM of S1 (Right in red). The proton current traces was monitored by evoking currents from a holding voltage of −60 mV with stepping to 0 mV for 1.5 s with a 10 s interpulse interval. According to FIG. 6, 100 nM concentration of S1 at −20 mV blocks 92% of the Hv1 channel current, indicating a significant increase in affinity of the S1 compound to the Hv1 channel as the result of the chemical change.

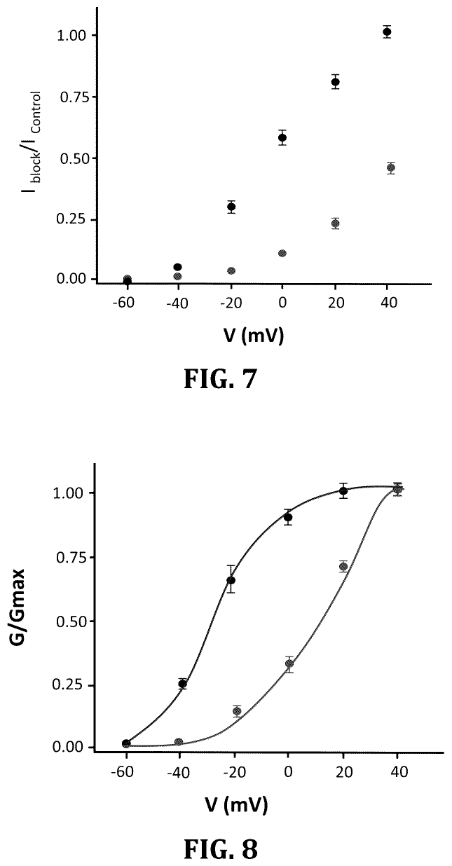

FIG. 7 illustrates a current-voltage relationships (I-V) for Hv1 in the absence (black) or presence of 100 nM of S1 (red). The current-voltage relationships were evoked from a holding potential of −60 mV to test pulses from −60 mV to +40 mV for 1.5 s in 20 mV intervals every 10 s. The fractional unblocked current was assessed at the end of the test pulse. As illustrated by the I-V plot, 100 nM concentration of S1 blocks 92% of Hv1 current at −20 mV with Ki of 3 nM. The inhibition constant Ki of S1 for Hv1 channels was calculated as Ki=Kon/Koff, wherein Kon is 9*10\hat{ }5 and Koff is 2*10\hat{ }5.

FIG. 8 illustrates a conductance-voltage relationships (G-V) for Hv1 in the absence (black) or presence of 100 nM of S1 (red). The GV shows about 41 mv V1/2 shift in voltage indicating S1 as a strong pore blocker with high affinity for the Hv1 channel. The conductance-voltage relationships were fit to the Boltzmann equation, G=Gmax/[1+exp(−zF(V−V1/2)/RT)], where V is the test potential, V1/2 is the voltage of half-maximal activation, z is the effective valence, T is the temperature, R is the gas constant, and F is the Faraday constant.

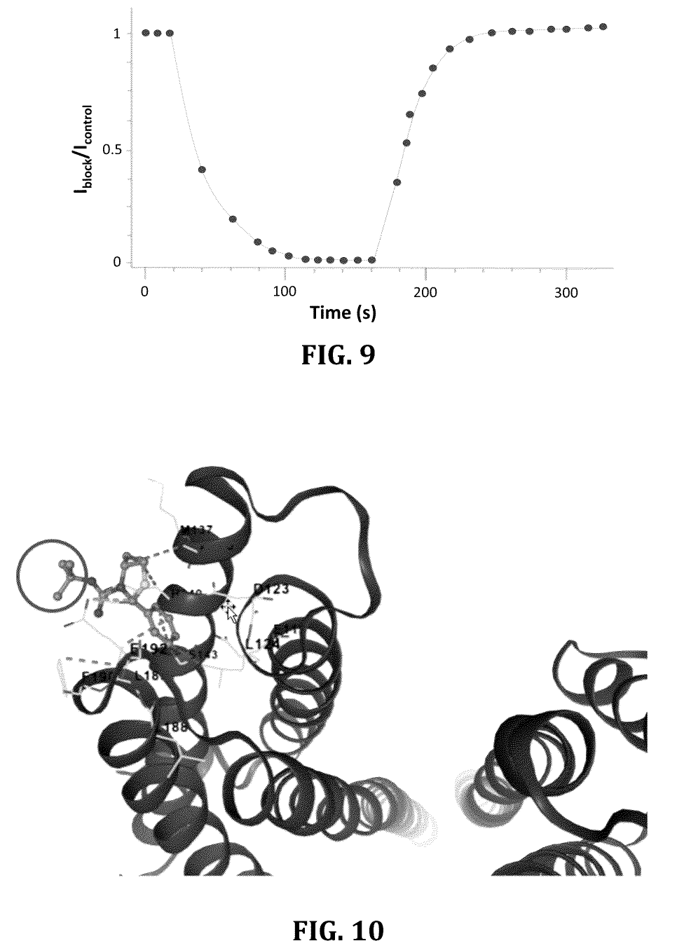

FIG. 9 illustrates a time course for block and unblock (washin and washout) of Hv1 when S1 is applied and when S1 is washed away. In FIG. 9, the Y axis shows the fractional current and the X axis shows the time course of block and unblock of the Hv1 channel. When 100 nM concentration of S1 is applied to Hv1 channels, it blocks the channel current immediately (washin), and the current resumes when the S1 is washed away with isotonic saline (washout). The curves were fit to the Hill equation, Fun=(1+([compound]/Ki)h)−1), wherein the [compound] is the effective compound concentration, Fun is the fraction of unblocked current at equilibrium. Ki is the inhibition constant and h is the Hill coefficient.

Based on the foregoing, unlike the Original Compound, the first substituent S1 provides a strong nano-molar pore blocker for Hv1 channels.

### Cryogenic-Electron Microscopy (Cryo-EM) Studies of S1

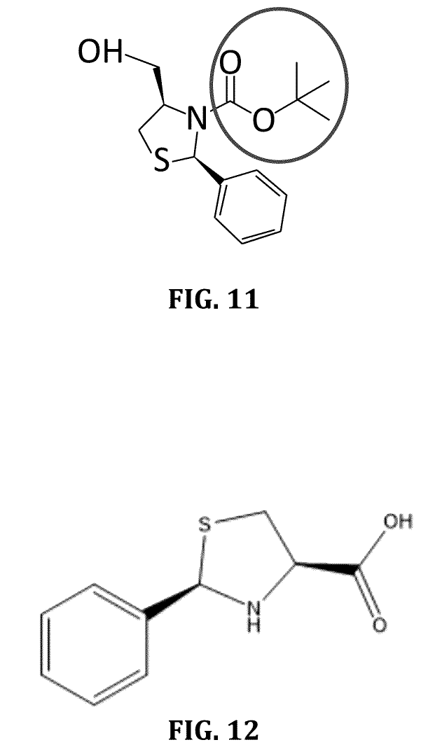

FIG. 10 shows the Cryogenic-Electron Microscopy (cryo-EM) data indicating how the S1 compound interacts with the Hv1 channel. The data identifies the important amino acids, binding sites, and side chains that are not involved in interaction with the Hv1 channel. According to the cryo-EM studies, two important amino acid residues, Methionine on position 137 (MET 137) and Glutamic acid on position 192 (GLU 192), were identified as the sites that the S1 compound interacted with the Hv1 channel. According to FIG. 11, cryo-EM studies further indicated that a side chain on S1 was not directly involved in interacting with Hv1 channel (highlighted in red circle). Based on this observation, by removing the side chain that was not involved in binding with the Hv1 channel, Inventor synthesized another substituent compound (S2) discussed below.

### Second Substituent Compound (S2)

FIG. 12 illustrates a second substituent compound (S2), having a chemical formula of (2R,4R)-2-phenylthiazolidine-4-carboxylic acid, that is synthesized from the first substituent compound (S1).

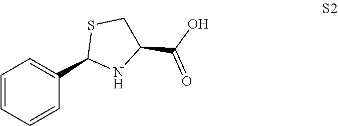 [SMILES: O=C(O)[C@@H]1CS[C@H](c2ccccc2)N1]

As discussed above with regard to FIGS. 10-11, the second substituent compound S2 can be made by removing the extra side chain of the first substituent S1 that is not involved in binding with the Hv1 channel.

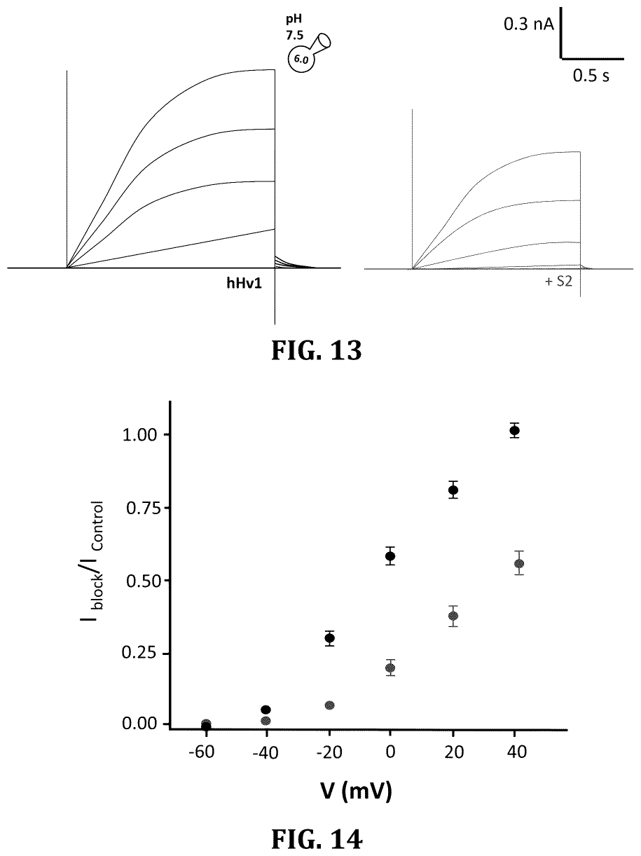

FIG. 13 illustrates representative proton current traces for Hv1 (hHv1) channels before (Left in black), and in the presence of 100 nM concentration of S2 (Right in red). The proton current traces was monitored by evoking currents from a holding voltage of −60 m V with stepping to 0 mV for 1.5 s with a 10 s interpulse interval. According to FIG. 13, 100 nM concentration of S2 at −20 mV blocks 85% of the Hv1 channel current with Ki of 20 nM, indicating the increase in affinity of the compound to the channel as a result of the chemical change to the Original Compound.

FIG. 14 illustrates a current-voltage relationships (I-V) for Hv1 in the absence (black) or presence of 100 nM of S2 (red). The current-voltage relationships were evoked from a holding potential of −60 mV to test pulses from −60 mV to +40 mV for 1.5 s in 20 mV intervals every 10 s. The fractional unblocked current was assessed at the end of the test pulse. As illustrated by the I-V plot, 100 nM concentration of S2 blocks 85% of Hv1 current at −20 mV with Ki of 20 nM. The inhibition constant Ki of S2 for Hv1 channels was calculated as Ki=Kon/Koff, wherein Kon is 7.5*10\hat{ }5 and Koff is 3.7*10\hat{ }4.

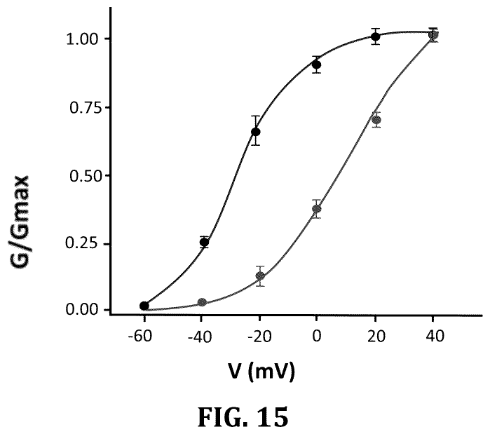

FIG. 15 illustrates a conductance-voltage relationships (G-V) for Hv1 in the absence (black) or presence of 100 nM of S2 (red). The GV shows about 35 mV V1/2 shift to the left which indicates that S1 is a relatively strong pore blocker of the Hv1 channel. The conductance-voltage relationships were fit to the Boltzmann equation, G=Gmax/[1+exp(−zF(V−V1/2)/RT)], where V is the test potential, V1/2 is the voltage of half-maximal activation, z is the effective valence, T is the temperature, R is the gas constant, and F is the Faraday constant.

Based on the foregoing, unlike the Original Compound, the second substituent S2 provides a strong nano-molar pore blocker for Hv1 channels.

Synthesis of S1 and S2 from the Original Compound

According to an implementation of the invention, the method of extracting S1 and S2 compounds from the marine fungi (Original Compound) may comprise the steps of using marine fungi samples that are preserved in ethanol, and performing solid liquid extraction using ethanol as solvent (Maceration). In Maceration, marine fungi samples are freeze-dried, then grounded to powder and soaked in solvent. The solvent is prepared to increase polarity. The solvent may consist of Hexane, Dichloromethane, methanol, and H2O. After the marine fungi soaked in solvent overnight, the soaked sample is filtered to obtain a filtrate. The filtrate is evaporated to obtain a crude extract. After obtaining the extract, a liquid-liquid extraction is performed followed by FPLC to identify the number of compounds. A HPLC technique may be further utilized for further fractionation and purification. After performing HPLC, the structure of the original marine fungi can be elucidated using NMR, and S1 and S2 can be synthesized using the original marine fungi as the backbone.

### Methods of Inhibiting Ion Transport by a Voltage-Gated Ion Channel, and Dosage

Certain embodiments of the present invention provide methods of inhibiting ion transport by a voltage-gated ion channel in a subject in need thereof by administering to the subject a therapeutically effective amount of a compound according to the present invention. The subject can be a mammal or a human. Such a subject may be in need of such methods for a variety reasons such as a disease and/or other problems associated with the ion transport activity of a voltage-gated ion channel. The targeted voltage-gated ion channel can be a proton channel, such as Hv1, and the ion transport activity can be proton transport. A non-limiting example of such a disease is Alzheimer's disease in human.

The methods of inhibiting ion transport may involve administering to the subject a therapeutically effective amount of a compound that is capable of inhibiting proton transport by a voltage-gated proton channel such as the Hv1 channel, wherein the compound can be the Original Compound (as shown in FIG. 1), S1 (as shown in FIG. 5), and/or S2 (as shown in FIG. 12).

Routes of administering a compound according to the present invention include, but not limited to oral, percutaneous, parenteral, and intravenous. And administering a therapeutically effective amount of a compound according to the present invention can be accomplished by administering an amount of the compound reasonably calculated to provide ion (e.g., proton) transport inhibiting levels of the compound to intended tissues and/or cells of the subject in need thereof.

Some embodiments of the present invention may provide pharmaceutical formulations that comprise a compound of the present invention together with excipients in solid or liquid dosage forms. Exemplary solid dosage forms include tablets, capsules, pills, and the like. Exemplary liquid dosage forms include injectable solutions, drinkable solutions, and the like.

In some embodiments, one or more compound(s) according to the present invention are co-administered with additional drugs or active agents that have voltage-gated ion channel inhibition activity. Such coadministration can comprise simultaneous administration or sequential administration within a time period selected from a week or less, a day or less, 12 hours or less, six hours or less, four hours or less, one hour less, in 15 minutes or less. Examples of such additional drugs or active agents include zinc, verapamil, 4-aminopyridine, PAP-1, correolide, TRAM-34, azimilide, imipramine, flecainide, and lamotrigine. In some embodiments, one or more compound(s) according to the present invention are coadministered with additional drugs or active agents that have inhibition activity on the NADPH oxidase, such as apocynin, VAS2870, ML171, GKT136901, celastrol.

According to Food and Drug Administration's Center for Drug Evaluation and Research (CDER), the following equation may provide a formula for converting animal doses to the human equivalent dose (HED) based on body weights and the allometric exponent (b):

HED=Animal NOAEL×(WeightAnimal/WeightHuman)^{(1-b)}

Wherein conventionally, for a mg/m2 normalization, b would be 0.67. (Guidance for Industry Estimating the Maximum Safe Starting Dose in Initial Clinical Trials for Therapeutics in Adult Healthy Volunteers; U.S. Department of Health and Human Services, Food and Drug Administration, Center for Drug Evaluation and Research (CDER); July 2005). For newly developed drug molecule, the NOAEL value in rat weighing approximately 150 g is 18 mg/kg. To calculate the starting dose for human studies, using the above equation, the HED would be 2.5 mg/kg. Accordingly, for a 60 kg human, the dose can be 150 mg. This HED value can be further divided by a factor value of 10. Thus, the initial dose in entry into man studies can be 15 mg. (See Nair A B, Jacob S., A simple practice guide for dose conversion between animals and human, J Basic Clin Pharm. 2016, doi: 10.4103/0976-0105.177703, PMID: 27057123; PMCID: PMC4804402). In various implementations of the invention, the exemplary amounts of the compound may include 1 mg/kg, 2.5 mg/kg, 5 mg/Kg, and 10 mg/Kg; and these values may be further divided by a factor value of 10 in entry to human studies.

The foregoing descriptions of embodiments of the present invention have been presented only for purposes of illustration and description. They are not intended to be exhaustive or to limit the present invention to the forms disclosed. Accordingly, many modifications and variations will be apparent to practitioners skilled in the art. Additionally, the above disclosure is not intended to limit the present invention. The scope of the present invention is defined by the appended claim.

### Experimental Studies, Cell Culture

For conducting experiments, we used HEK293T cells. The HEK293T cells were purchased from American Type Culture Collection (ATCC). For cell culture, the HEK293T cells were maintained in ATTC's Dulbecco's Modified Eagle's Medium (DMEM) supplemented with a 10% fetal bovine serum (FBS 10%) and 1% penicillin and streptomycin. Plasmids were transfected into cells using a transfection reagent, Lipofectamine™ 2000 according to the manufacturer's instructions (Life Technologies). Experiments were performed 24-48 hours post-transfection.

### Experimental Studies, Whole Cell Patch-Clamp Method to Study Hv1 Proton Currents

For the experimental studies, similar techniques as utilized by Zhao et. al is used in this invention. (Zhao et al., Role of human Hv1 channels in sperm capacitation and white blood cell respiratory burst established by a designed peptide inhibitor, Proc Natl Acad Sci USA. 2018, PMID: 30478045, PMCID: PMC6294887).

In the present invention, a Whole Cell patch clamp technique was utilized to study the Hv1 proton currents. The proton currents passed by Hv1 were recorded in Whole Cell mode using an Axon™ Axopatch™ 200B microelectrode amplifier. Stimulation and data collection were performed by a high-resolution, low-noise digitizer, Digidata 1322A, manufactured by Axon Instruments, Inc.) and Axon™ pCLAMP™ 10 Electrophysiology Data Acquisition & Analysis Software (released by Molecular Devices). Cells were perfused with an external bath solution comprised of a zwitterionic sulfonic acid buffering agent (100 mM HEPES), 100 mM NaCl, and 10 mM glucose at pH 7.5. During the patch clamp recording, the patch pipettes with a resistance between 3 and 5 MΩ were filled with 100 mM Bis-Tris buffer, 100 mM NaCl, and 10 mM glucose at pH 6.0. The sampling frequency was 10 kHz and the signals were filtered at 1 KHz.

The compound block (proton current traces as shown in FIGS. 2, 6, and 13) was monitored by evoking currents from a holding voltage of −60 mV with stepping to 0 mV for 1.5 s with a 10 s interpulse interval.

The current-voltage relationships (I-V plots as shown in FIGS. 3, 7, and 14) were similarly evoked from a holding potential of −60 mV to test pulses from −60 mV to +40 mV for 1.5 s in 20 mV intervals every 10 s. Fractional unblocked current was assessed at the end of the test pulse.

The conductance-voltage relationships (G-V plots as shown in FIGS. 4, 8, and 15) were fit to the Boltzmann equation, G=Gmax/[1+exp(−zF (V−V1/2)/RT)], where V is the test potential, V1/2 is the voltage of half-maximal activation, z is the effective valence, T is the temperature, R is the gas constant, and F is the Faraday constant.

Dose-response curves (the time course for block and unblock of Hv1 as shown in FIG. 9) show the fractional unblocked current, Icompound/Icontrol, versus toxin concentration. The curves were fit to the Hill equation, Fun=(1+([compound]/Ki)h)−1), wherein the [compound] is the effective compound concentration, Fun is the fraction of unblocked current at equilibrium. Ki is the inhibition constant and h is the Hill coefficient.

What is claimed is:

1. A composition of matter, comprising a molecule of the structure I:

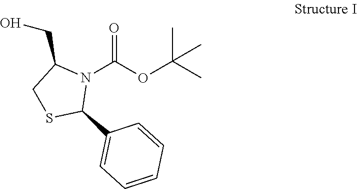 [SMILES: CC(C)(C)OC(=O)N1[C@H](CO)CS[C@@H]1c1ccccc1]

2. The composition of matter of claim 1, wherein the structure I is synthesized from marine fungi.

3. The composition of matter of claim 2, wherein the composition inhibits proton transport by Hv1 channels in living tissues.

4. A composition of matter, comprising a molecule of the structure II:

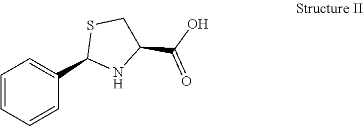 [SMILES: O=C(O)[C@@H]1CS[C@H](c2ccccc2)N1]

5. The composition of matter of claim 4, wherein the structure II is synthesized from marine fungi.

6. The composition of matter of claim 5, wherein the composition inhibits proton transport by Hv1 channels in living tissues.

7. A method of inhibiting proton transport by a Hv1 channel comprising:
administering to a subject a therapeutically effective amount of a pharmaceutical composition that is capable of inhibiting proton transport by the Hv1 channel;
wherein the pharmaceutical composition comprises a molecule of the structure I or the structure II:

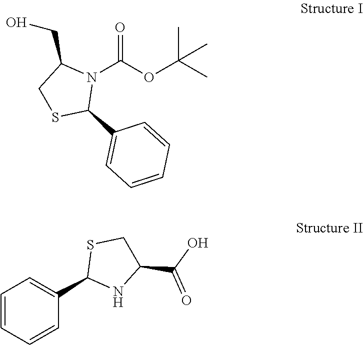 [SMILES: CC(C)(C)OC(=O)N1[C@H](CO)CS[C@@H]1c1ccccc1 ; O=C(O)[C@@H]1CS[C@H](c2ccccc2)N1]

8. The method of claim 7, wherein the subject is a human and the therapeutically effective amount is 2.5 mg/kg of the weight of the human.
